(19) United States
(12) Plant Patent Application Publication
(10) Pub. No.: US 2015/0289431 P1
(43) Pub. Date: Oct. 8, 2015

(54) Strawberry plant named 'NJ99-204-1'

(50) ;

(71) Applicant: Rutgers, The State University, New Brunswick, NJ (US)

(72) Inventors: Gojko J. Jelenkovic, Piscataway, NJ (US); Laurie P. Lutz, Rockville, MD (US); Peter J. Nitzsche, Long Valley, NJ (US); William T. Hlubik, Bordentown, NJ (US)

(73) Assignee: Rutgers, The State University, New Brunswick, NJ (US)

(21) Appl. No.: 13/999,932

(22) Filed: Apr. 4, 2014

(51) Int. Cl. A01H 5/00

(52) U.S. Cl. PLT/208

(57) ABSTRACT

A new and distinct cultivar of strawberry plant (Fragaria×annanassa), was developed from seed produced by a hand pollinated cross between ‘NJ 9612-1’ and ‘Camarosa’ (U.S. Plant Pat. No. 8,708). The new strawberry named ‘NJ99-204-1’ is distinguished by its ability to produce large, uniform, and firm fruit with excellent flavor. These vigorous plants have an upright form.

### CROSS REFERENCE TO RELATED APPLICATIONS

NONE

### STATEMENT REGARDING FEDERALLY SPONSORED RESEARCH OR DEVELOPMENT

NONE

### LATIN NAME OF GENUS AND SPECIES OF THE PLANT CLAIMED

Fragaria annanassa

### VARIETY DENOMINATION

STRAWBERRY PLANT NAMED ‘NJ99-204-1’

### BACKGROUND OF THE NEW PLANT

A new and distinct cultivar of strawberry (Fragaria×annanassa) named ‘NJ99-204-1’ is a short day cultivar similar to ‘Chandler’ (U.S. Plant Pat. No. 5,262) but it is distinguished by its large berry size, long wedge to long conical shape, uniform deep red color, and exceptional flavor. The cultivar is well adapted to high density plasticulture growing systems and has been shown to perform well in the eastern United States. (Zones 5b, 6a & b, 7a & b, 8a & b, and 9a) This new variety should be of commercial value, particularly for farmers with direct markets, due to its unique fruit characteristics and excellent flavor.

### ORIGIN OF THE VARIETY

This new strawberry genotype was developed from a controlled cross of NJ 9612-1 (unpatented) as the female parent and the commercial strawberry variety ‘Camarosa’ (U.S. Plant Pat. No. 8,708) as the male parent. Seeds of the cross were germinated in a greenhouse and planted in the field at the NJAES research farm #3 in New Brunswick, N.J. in 1999. The selected seedling designated as ‘NJ99-204-1’, was recognized for its vigor, healthy phenotype, productivity and superior fruit quality. The ‘NJ99-204-1’ plant was then asexually propagated for further evaluations from 2000 to 2012 in observational and replicated trials in several locations in New Jersey and one location in North Carolina.

### SUMMARY OF THE VARIETY

‘NJ99-204-1’ is primarily adapted to the climate and conditions of the eastern United States where it demonstrates vigorous plant growth. It is characterized by its production of large, uniformly shaped, long wedge and conical fruit, with exceptional flavor.

### BRIEF DESCRIPTION OF THE PHOTOGRAPHS

FIG. 1 illustrates the initial stages of typical flowers and fruit developing on a ‘99-204-1’ plant in the early spring;

FIG. 2 illustrates plant and fruit characteristics after the fruit has matured on the ‘99-204-1’ plant; and

FIG. 3 illustrates the characteristics (shape, size and coloring) of typical ripened fruit from a ‘99-204-1’ plant.

All color references below are measured against The Royal Horticultural Society Colour Chart (R.H.S. 1995 Ed.). Colors of foliage, fruit, inflorescence and other plant parts may vary from year to year depending on horticultural practices, light conditions, air temperature, soil fertility, etc.

### DETAILED BOTANICAL DESCRIPTION

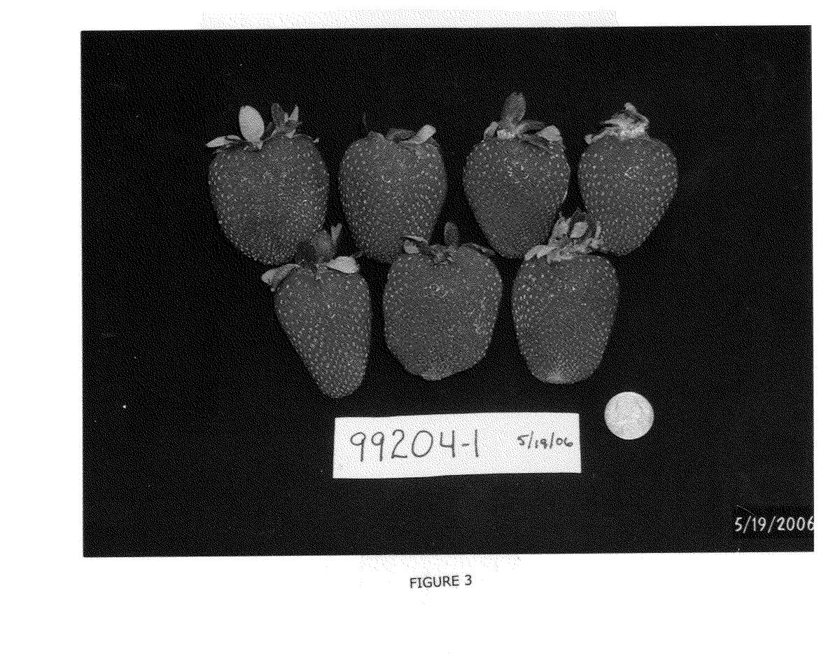

The ‘NJ99-204-1’ plant can be distinguished from other strawberry varieties by assessing the development pattern and appearance of various parts of the plant during growing and harvesting season. The primary fruits are usually wedge shaped with uplifted sepals and conspicuous fruit knack. The fruit color is dark red (RHS 45A (FIG. 3). The secondary and tertiary fruits are predominantly conic in shape. The fruits are only slightly longer than they are wide with a 1.07:1 length to width ratio. The aromatic taste and pleasant texture of the fruits when consumed at their full maturity are important identification markers.

The development of both uniped and branched inflorescence is perhaps the most characteristic attribute of this genotype. The petal color of the flowers are white (RHS 155C). The leaf petioles show upright directional growth rather than prostrate; this leads to the formation of a symmetrical bushy canopy of leaves at the top of the plant. The lobes of trifoliate leaves are of medium size, oblong in shape, leathery in structure with a presence of numerous hairs on both the adaxial and abaxial sides. The color of upper surface (adaxial) is RHS 141 C and the color of the abaxial surface is RHS 141D. The leaf is cup-shaped and the margins are serrated.

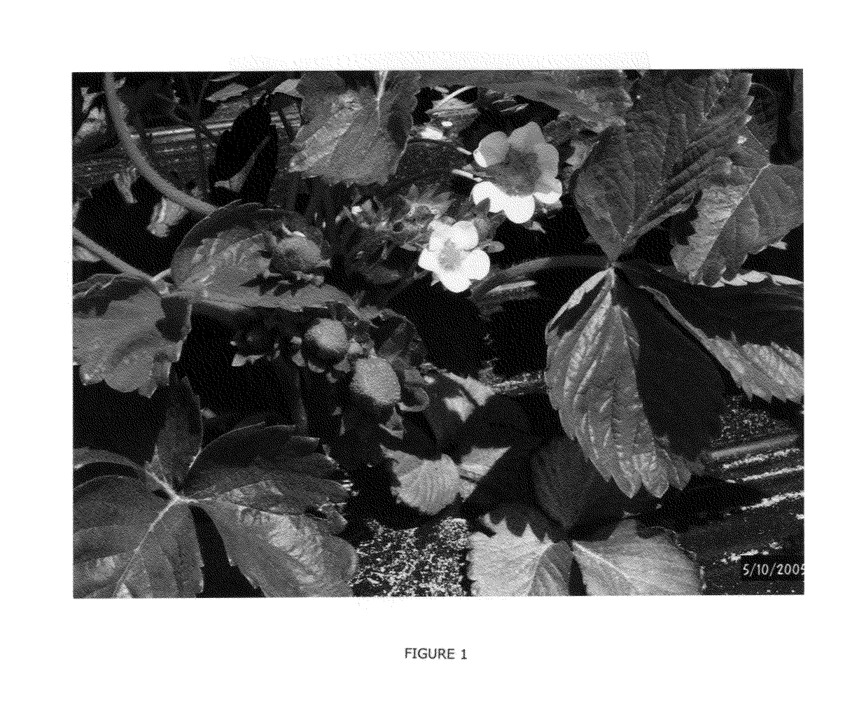

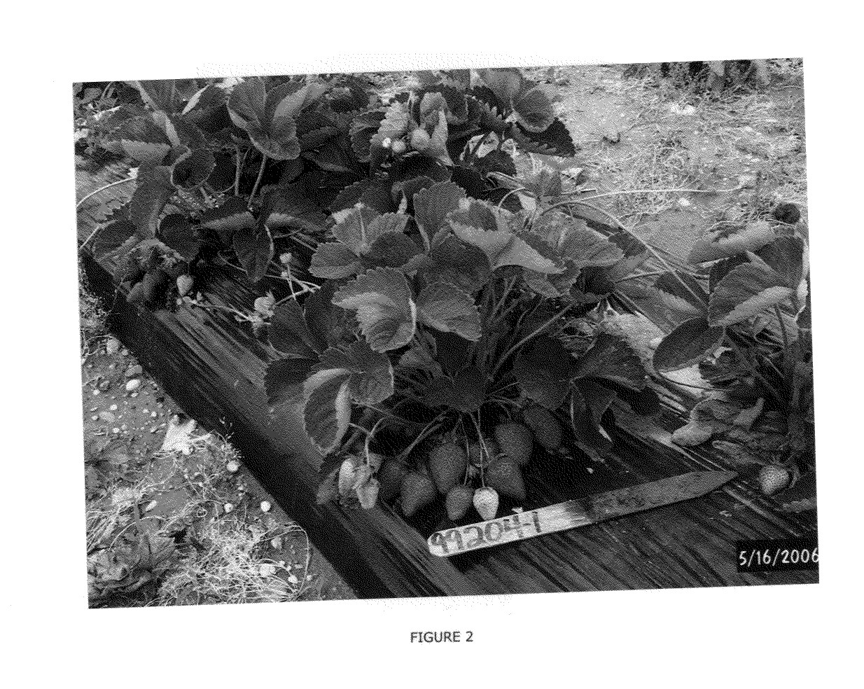

Structurally, two different types of inflorescences are found. In the first type, the peduncle branches into 4-8 pedicles, each one bearing a single flower. The branching may take place shortly after peduncle emerges from bud; short peduncle branching, or after a peduncle elongates quite a distance; long peduncle branching. The central pedicel is strongest and eventually will produce the largest fruit—the primary fruit, the rest of pedicles are weaker and will produce smaller size fruits—secondary, tertiary, etc. (FIG. 2). In the second type, the branching of the peduncle takes place early in its development, before emerging from the bud and each pedicle is terminated by a single flower. The pedicles are fewer in number per inflorescence but much stronger and, as consequence, the primary, secondary and tertiary are larger in size in this type of inflorescence (FIG. 1). Both types of inflorescence could develop on the same plant; however, the branching type seems to be predominant later in the spring. The hermaphroditic flowers develop with great regularity, containing 12-18 plump anthers loaded with well developed functional pollen grains. In regard to the pollination process, observations in various seasons and locations of growth have indicated that this process is regular, leading to formation of well shaped attractive fruits.

From the time of flowering to the time of fruit ripening (harvesting) it normally takes about 28-34 days; which categorizes this genotype as a mid-season variety. Fruits turn red acropetally, from the fruit knack toward the tip. Under particularly warm conditions in the spring, a primary fruit can become completely red in a single day; however, formation of anthogenesis, sugars, aromatics and other quality ingredients require at least two additional days.

TABLE 1
Field performance of NJAES strawberry selections,
Pittstown, NJ 2011

| | ‘NJ99-204-1’ | Chandler | Avalon* |
|---|---|---|---|
| Marketable yield (lb/A) | 3,474 a | 4,823 a | 3,747 a |
| Average fruit size (g)^{y} | 18.94 a | 15.32 b | 16.56 ab |

^{y}Twenty representative fruit/plot
zMean separation within columns by LSD, α = 0.05
*U.S. Plant Patent No. 11,372

TABLE 2
Field Performance of NJAES strawberry selections and cultivars, Salisbury, NC 2010

| | | | | Florida | |
|---|---|---|---|---|---|
| | ‘NJ99-204-1’^{z} | Chandler^{z} | Camarosa^{^{z}} | Radiance^{z}* | Galletta^{z}** |
| Marketable yield (lb/A) | 22,756 b | 28,352 a | 19,363 bc | 14,816 cd | 12,775 d |
| Average fruit size (g)^{y} | 23.5 cd | 21.9 cd | 24.1 bc | 23.0 cd | 26.1 b |
| % Soluble Solids (^{o}Brix^{x}) | 8.00 ab | 7.16 cd | 7.67 be | 5.98 f | 7.75 abc |

^{x}Average of samples from Apr. 29, May 3, May 5, 2010 harvests
^{y}Twenty five representative fruit/plot
^{z}Mean separation within columns by LSD, α = 0.05
*U.S. Plant Patent No. 20,363
**U.S. Plant Patent No. 19,763.

TABLE 3
Flower Characteristics of NJAES strawberry selections and
cultivars, North Brunswick, NJ 2013

| | ‘NJ99-204-1’^{x} | Chandler^{y} | Camarosa^{z} |
|---|---|---|---|
| Flower color | White | White | White |
| Flower Head diameter (mm) | 3.58 | 4.8 | 3.67 |
| Petal Number | 5.25 | 5.6 | 5.22 |
| Corolla diameter | 26 | 26.15 | 22.78 |
| Calyx diameter | 19.75 | 21.15 | 20.11 |
| Sepal Number | 10.75 | 11.2 | 10.89 |

Data taken May 22 to Jun. 15, 2013
^{x}Means based on the average of 12 observations.
^{y}Means based on the average of 20 observations.
^{z}Means based on the average of 9 observations.

TABLE 4
Fruit Characteristics of NJAES strawberry selections and
cultivars, NorthBrunswick, NJ 2013

| | | | Earliglow^{z} |
|---|---|---|---|
| | ‘NJ99-204-1’^{x} | Chandler^{y} | (Unpatented) |
| Fruit Length (mm) | 34.06 | 31.04 | 23.69 |
| Fruit Width (mm) | 32.04 | 26.41 | 22.77 |
| Length/Width Ratio | 1.07:1 | 1.18:1 | 1.04:1 |
| % Soluble Solids (brix) | 8.35 | 8.26 | 10.49 |
| Fruit Density (g) | 181.58 | 172.6 | 128 |
| Average Berry Weight | 14.09 | 12.37 | 8.59 |
| (g) | | | |

Data taken May 17 to Jun. 15, 2013
^{x}Means based on the average of 20 observations.
^{y}Means based on the average of 15 observations.
^{z}Means based on the average of 16 observations.

We claim:

1. A new and distinct strawberry plant named ‘NJ99-204-1’ as herein illustrated and described.
